(19) United States
(12) Patent Application Publication
(10) Pub. No.: US 2021/0217523 A1
(43) Pub. Date: Jul. 15, 2021

(54) DIAGNOSIS ASSISTANCE SYSTEM AND DIAGNOSIS ASSISTANCE DEVICE

(71) Applicants: Shimadzu Corporation, Kyoto (JP); The University of Tokyo, Tokyo (JP)

(72) Inventors: Kentaro MORIMOTO, Kyoto (JP); Masaya SATO, Tokyo (JP); Yutaka YATOMI, Tokyo (JP); Ryosuke TATEISHI, Tokyo (JP); Kazuhiko KOIKE, Tokyo (JP)

(21) Appl. No.: 17/056,232

(22) PCT Filed: Apr. 15, 2019
(86) PCT No.: PCT/JP2019/016122

§ 371 Date: Jan. 18, 2021

(30) Foreign Application Priority Data
May 18, 2018 (JP) 2018-096192

(51) Int. Cl. G16H 50/20; G16H 50/70; G16H 10/60; G06N 3/04

(52) U.S. Cl. CPC G16H 50/20; G06N 3/0454; G16H 10/60; G16H 50/70

(57) ABSTRACT

A diagnosis assistance system (100) includes a determiner (21) configured to receive trained model information (M) generated by a trained model generator (11) via an external network (30), the determiner (21) being configured to determine, based on the received trained model information (M), a presence or absence of a disease in a patient (P2) who is not included in a patient group (PF).

### TECHNICAL FIELD

The present invention relates to a diagnosis assistance system and a diagnosis assistance device, and more particularly, it relates to a diagnosis assistance system and a diagnosis assistance device, each of which includes a trained model generator configured to generate trained model information by machine learning based on biological information about a patient group.

### BACKGROUND ART

Conventionally, a diagnosis assistance device including a trained model generator configured to generate trained model information by machine learning based on biological information about a patient group is known. Such a diagnosis assistance device is disclosed in Japanese Patent Laid-Open No. 2018-041434, for example.

Japanese Patent Laid-Open No. 2018-041434 discloses a diagnosis assistance device configured to diagnose a lesion from a captured image. In this diagnosis assistance device, machine learning (neural network) is performed by an ensemble classifier (trained model generator). Specifically, a plurality of learning images (reference images) with a known presence or absence of a lesion are prepared for machine learning. Then, a predetermined image is extracted from the plurality of learning images, and a plurality of images having different rotation angles or magnifications, for example, of the extracted image are prepared. Then, these images are input to the ensemble classifier (neural network), and machine learning is performed. Consequently, a learned ensemble classifier is generated. Then, an image with an unknown presence or absence of a lesion is input to the learned ensemble classifier, and the presence or absence of a lesion is inferred (determined).

### PRIOR ART

### Patent Document

Patent Document 1: Japanese Patent Laid-Open No. 2018-041434

### SUMMARY OF THE INVENTION

### Problems to be Solved by the Invention

In machine learning such as neural network as disclosed in Japanese Patent Laid-Open No. 2018-041434, a relatively large number of images for machine learning (data for machine learning) are required. For example, in a relatively large hospital (large hospital), a large number of patients visit the hospital, and thus sufficient data for machine learning to perform machine learning can be acquired. On the other hand, in a relatively small hospital (small hospital), a small number of patients visit the hospital, and thus it is difficult to acquire sufficient data for machine learning to perform machine learning. In addition, the data for machine learning includes personal information for identifying patients, and the data for machine learning held in a large hospital cannot be used in a small hospital. Therefore, it is difficult to perform machine learning about the presence or absence of a lesion in a small hospital, and it is difficult to infer (determine) the presence or absence of a lesion based on the result of machine learning in a small hospital.

The present invention is intended to solve at least one of the above problems. The present invention aims to provide a diagnosis assistance system and a diagnosis assistance device, each of which is capable of determining the presence or absence of a disease in a patient based on the result of machine learning even in a small hospital or the like in which it is difficult to acquire data for machine learning, without leaking patient's personal information to the outside (such as the small hospital).

### Means for Solving the Problems

In order to attain the aforementioned object, a diagnosis assistance system according to a first aspect of the present invention includes a storage configured to store biological information about a patient group, a trained model generator configured to generate trained model information derived from a pattern included in the biological information about the patient group, by machine learning based on the biological information about the patient group stored in the storage, and a determiner configured to receive the trained model information generated by the trained model generator via an external network, the determiner being configured to determine, based on the received trained model information, a presence or absence of a disease in a patient who is not included in the patient group.

As described above, the diagnosis assistance system according to the first aspect of the present invention includes the determiner configured to receive the trained model information generated by the trained model generator via the external network and to determine the presence or absence of the disease in the patient who is not included in the patient group based on the received trained model information. Accordingly, the learn model information can be received by the determiner installed in a small hospital or the like via the external network 30, and thus even in a small hospital or the like in which it is difficult to acquire data for machine learning, the presence or absence of the disease in the patient can be determined based on the trained model information. Furthermore, the trained model information generated by the trained model generator includes statistical information, for example, that does not include patient's personal information. Thus, even when the trained model information is provided to the outside (such as a small hospital) via the external network, the patient's personal information is not leaked. Consequently, even in a small hospital in which it is difficult to acquire data for machine learning, for example, the patient's personal information is not leaked to the outside (such as a small hospital), and the presence or absence of the disease in the patient can be determined based on the result (trained model information M) of machine learning.

In the aforementioned diagnosis assistance system according to the first aspect, the storage is preferably configured to store electronic clinical record data with identification information about an individual patient included in the patient group and the biological information about the individual patient being described therein, and the trained model generator is preferably configured to extract the biological information about the individual patient from the electronic clinical record data stored in the storage, and to generate the trained model information based on the extracted biological information about the individual patient. Accordingly, the trained model information is generated based only on the biological information about the individual patient extracted from the electronic clinical record data without using the patient's identification information, and thus leakage of the patient's personal information can be reliably prevented.

In this case, the trained model generator is preferably configured to generate the trained model information based on the biological information about the individual patient extracted from the electronic clinical record data stored in the storage, and analytical information about a biological sample of the individual patient associated with the electronic clinical record data. Accordingly, the trained model information is generated based on the analytical information about the biological sample of the individual patient in addition to the biological information about the individual patient, and thus the presence or absence of the disease in the patient can be more accurately determined.

In the aforementioned diagnosis assistance system according to the first aspect, the determiner preferably includes a first trained model update unit configured to update the trained model information received via the external network based on the biological information about a patient with a known presence or absence of the disease, who is not included in the patient group. Accordingly, even when it is difficult to acquire data for machine learning, the quality (determination ability) of the trained model information can be improved based on the biological information about the patient with the known presence or absence of the disease, who is not included in the patient group.

The aforementioned diagnosis assistance system according to the first aspect preferably further includes a transmitter configured to transmit the biological information about a patient with a known presence or absence of the disease, who is not included in the patient group, to the trained model generator via the external network in a state in which identification information about the patient has been removed from the biological information, and the trained model generator preferably includes a second trained model update unit configured to update the trained model information based on the biological information transmitted from the transmitter. Accordingly, the quality (determination ability) of the trained model information can be improved by the second trained model update unit based on the biological information about the patient with the known presence or absence of the disease, who is not included in the patient group, transmitted from the outside (such as a small hospital) via the external network. Consequently, the trained model information with improved quality (determination ability) is received by a plurality of external facilities such as small hospitals again via the external network such that the presence or absence of the disease in the patient can be determined based on the common trained model information with improved quality in the plurality of external facilities. Furthermore, the transmitter transmits the biological information about the patient to the second trained model update unit in a state in which the identification information about the patient has been removed from the biological information, and thus the identification information about the patient is not leaked to the outside.

In the aforementioned diagnosis assistance system according to the first aspect, the trained model information is preferably exported from the trained model generator and imported into the determiner via the external network. Accordingly, even when the application of the trained model generator and the application of the determiner are different from each other, the trained model information is output (exported) from the trained model generator in a format that can be read by the application of the determiner such that the trained model information can be used in the determiner.

In the aforementioned diagnosis assistance system according to the first aspect, the biological information about the patient group preferably includes data on a presence or absence of liver cancer, HCV antibody, HBs antigen, age, gender, height, weight, albumin, total bilirubin, AST, ALT, ALP, GGT, a platelet, AFP, an L3 fraction, and DCP, and the determiner is preferably configured to determine the presence or absence of liver cancer in the patient who is not included in the patient group based on the trained model information received via the external network. Accordingly, the presence or absence of liver cancer in the patient can be determined with a relatively high correct answer rate. It has been confirmed by an experiment conducted by the inventors described below that the presence or absence of liver cancer can be determined with a relatively high correct answer rate based on the above biological information.

A diagnosis assistance device according to a second aspect of the present invention includes a storage configured to store biological information about a patient group, and a trained model generator configured to generate trained model information derived from a pattern included in the biological information about the patient group, by machine learning based on the biological information about the patient group stored in the storage. The trained model information generated by the trained model generator is exported to an outside via an external network.

As described above, the diagnosis assistance device according to the second aspect of the present invention is configured to export the trained model information generated by the trained model generator to the outside via the external network. Accordingly, the learn model information can be received by a determiner installed in a small hospital or the like via the external network, and thus even in a small hospital or the like in which it is difficult to acquire data for machine learning, the presence or absence of a disease in a patient can be determined based on the trained model information. Furthermore, the trained model information generated by the trained model generator includes statistical information, for example, that does not include patient's personal information. Thus, even when the trained model information is provided to the outside (such as a small hospital) via the external network, the patient's personal information is not leaked. Consequently, it is possible to provide the diagnosis assistance device capable of determining the presence or absence of the disease in the patient based on the result (trained model information) of machine learning even in a small hospital or the like in which it is difficult to acquire data for machine learning, without leaking the patient's personal information to the outside (such as the small hospital).

In aforementioned the diagnosis assistance device according to the second aspect, the storage is preferably configured to store electronic clinical record data with identification information about an individual patient included in the patient group and the biological information about the individual patient being described therein, and the trained model generator is preferably configured to extract the biological information about the individual patient from the electronic clinical record data stored in the storage, and to generate the trained model information based on the extracted biological information about the individual patient. Accordingly, the trained model information is generated based only on the biological information about the individual patient extracted from the electronic clinical record data without using the patient's identification information, and thus leakage of the patient's personal information can be reliably prevented.

In this case, the trained model generator is preferably configured to generate the trained model information based on the biological information about the individual patient extracted from the electronic clinical record data stored in the storage, and analytical information about a biological sample of the individual patient associated with the electronic clinical record data. Accordingly, the trained model information is generated based on the analytical information about the biological sample of the individual patient in addition to the biological information about the individual patient, and thus the presence or absence of the disease in the patient can be more accurately determined.

### Effect of the Invention

According to the present invention, as described above, it is possible to determine the presence or absence of the disease in the patient based on the result of machine learning even in a small hospital or the like in which it is difficult to acquire data for machine learning, without leaking the patient's personal information to the outside (such as the small hospital).

### BRIEF DESCRIPTION OF THE DRAWINGS

FIG. 1 is a block diagram of a diagnosis assistance system according to a first embodiment of the present invention.

FIG. 2 is a diagram for illustrating the diagnosis assistance system according to the first embodiment of the present invention.

FIG. 3 is a block diagram of a diagnosis assistance system according to a second embodiment of the present invention.

FIG. 4 is a block diagram of a diagnosis assistance system according to a third embodiment of the present invention.

FIG. 5 is a diagram for illustrating the diagnosis assistance system according to the third embodiment of the present invention.

FIG. 6 is a block diagram of a diagnosis assistance system according to a fourth embodiment of the present invention.

### MODES FOR CARRYING OUT THE INVENTION

Embodiments embodying the present invention are hereinafter described on the basis of the drawings.

### First Embodiment

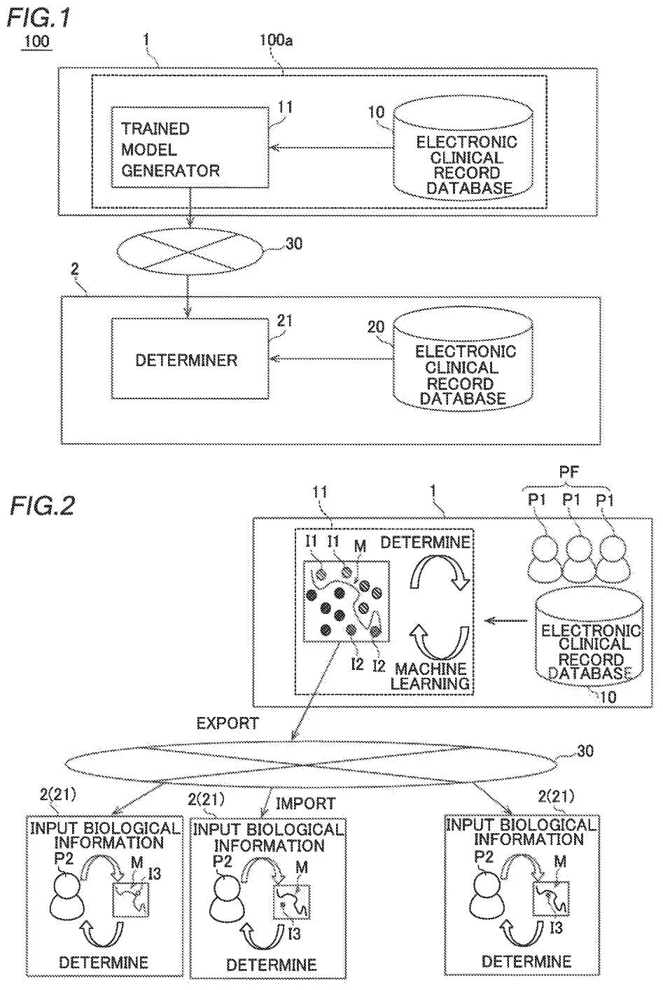

The configuration of a diagnosis assistance system 100 according to a first embodiment is now described with reference to FIGS. 1 and 2.

First, an electronic clinical record (electronic clinical record data) is described. The electronic clinical record (electronic clinical record data) is for electronically storing a doctor's diagnosis record instead of paper (information system). The electronic clinical record allows the clerical work of medical staffs to be streamlined and allows information management to be unified. The results obtained in an examination facility in which a patient P1 is examined can be automatically linked to the electronic clinical record. Furthermore, in the electronic clinical record, the visibility of the characters is good, and the electronic clinical record can be easily searched.

In diagnosis using a conventional clinical record (such as an electronic clinical record), a doctor comprehensively judges the condition of the patient P1 from information obtained by the electronic clinical record and the interview, and makes a diagnosis. This diagnosis refers to performing a more burdensome and highly invasive examination on the patient P1 and determining a treatment policy. In addition, the doctor's comprehensive judgment is based on the doctor's experience backed by statistical knowledge. On the other hand, the diagnosis assistance system 100 according to the first embodiment makes a diagnosis (assists a diagnosis) based on a pattern (trained model information M) included in biological information about a patient group PF.

As shown in FIG. 1, the diagnosis assistance system 100 includes an electronic clinical record database 10, a trained model generator 11, an electronic clinical record database 20, and a determiner 21. The electronic clinical record database 10 and the trained model generator 11 are arranged in a facility 1 such as a large hospital that a relatively large number of patients visit. The electronic clinical record database 20 and the determiner 21 are arranged in a facility 2 such as a small hospital that a small number of patients visit. The electronic clinical record database 10 and the trained model generator 11 are provided in a diagnosis assistance device 100a. The trained model generator 11 and the determiner 21 include software (programs). The electronic clinical record database 10 is an example of a “storage” in the claims.

The electronic clinical record database 10 stores the biological information about the patient group PF. Specifically, the electronic clinical record database 10 stores electronic clinical record data with identification information (name, etc.) about individual patients P1 included in the patient group PF and biological information about the individual patients P1 being described therein. The biological information about the patient group PF includes data on the presence or absence of liver cancer, HCV antibody, HBs antigen, age, gender, height, weight, albumin, total bilirubin, AST, ALT, ALP, GGT, platelets, AFP, L3 fractions, and, DCP. HCV antibody is an indicator indicating whether the patient was once infected with hepatitis C virus or is currently persistently infected with hepatitis C virus. HBs antigen is an indicator indicating that hepatitis B virus is currently present (the patient is infectious). Albumin is a numerical value obtained by measuring the concentration of protein in serum, and abnormalities in the liver and kidneys can be investigated based on a decrease in albumin. Total bilirubin is an indicator of the metabolic capacity of the liver. AST is an indicator to know how much damage is occurring mainly in the liver and heart. ALT is an indicator to know whether the liver is damaged. GGT is an indicator of liver function. AFP is an indicator of the presence or absence of liver cancer. The L3 fraction is a value indicating how much AFP-L3 is contained in AFP. DCP is an abnormal prothrombin that is synthesized in the liver and has no coagulation activity, and is a tumor marker specific to hepatocellular carcinoma.

The trained model generator 11 is configured to generate the trained model information M derived from a pattern included in the biological information about the patient group PF, by machine learning based on the biological information about the patient group PF stored in the electronic clinical record database 10. Specifically, in the first embodiment, the trained model generator 11 is configured to extract the biological information about the individual patients P1 from the electronic clinical record data stored in the electronic clinical record database 10 and to generate the trained model information M based on the extracted biological information about the individual patients P1.

Machine learning is to repeatedly learn training data (data of a known determination result) and find a pattern hidden in the training data. In the machine learning, various algorithms are used to repeatedly learn the training data, and thus even when where humans should search (a portion of the training data) is not explicitly programmed, a computer autonomously derives the pattern. In this specification, the pattern found by machine learning is referred to as the trained model information M. When certain data (in the first embodiment, the electronic clinical record data of the facility 2 described below) is applied (input) to the trained model information M, the presence or absence of liver cancer is determined based on the learned pattern.

Personal information such as the names of the patients P1 is described in the electronic clinical record data. In addition, the biological information (data on the presence or absence of liver cancer, HCV antibody, HBs antigen, age, gender, height, weight, albumin, total bilirubin, AST, ALT, ALP, GGT, platelets, AFP, L3 fractions, and, DCP) is described in the electronic clinical record data. The trained model generator 11 extracts the biological information (data on the presence or absence of liver cancer, HCV antibody, HBs antigen, age, gender, height, weight, albumin, total bilirubin, AST, ALT, ALP, GGT, platelets, AFP, L3 fractions, and, DCP) from the electronic clinical record data.

The trained model generator 11 uses machine learning using a logistic regression to create linear trained model information M, or a soft-margin support vector machine, a neural network, a random forest, or the like to create non-linear trained model information M to generate the trained model information M. The created trained model information M has properties close to numerical information that has been statistically processed, and is data that does not include the personal information about the patients P1.

In the logistic regression, a variable to be predicted is referred to as an objective variable (in the first embodiment, the presence or absence of liver cancer). In addition, a variable that affects the objective variable is referred to as an explanatory variable (in the first embodiment, the biological information). In the logistic regression, the relationship between the objective variable and the explanatory variable is expressed by a relational expression. The logistic regression is to calculate a predicted value (a predicted value for the presence or absence of liver cancer) using the above relational expression, and determine the degree of contribution of the explanatory variable used in the relational expression to the objective variable.

The support vector machine finds a hyperplane that separates some pieces of biological information (feature amount) of the presence of liver cancer from some pieces of biological information (feature amount) of the absence of liver cancer when training data (in the first embodiment, the biological information) is given. In addition, the support vector machine finds a maximum-margin hyperplane among a plurality of hyperplanes that separate biological information. The margin refers to a minimum value of a distance between the hyperplane and each feature point, and a hyperplane that maximizes this margin is found. A method for finding a hyperplane that completely separates feature points of the presence of liver cancer from feature points of the absence of liver cancer is called a hard-margin support vector machine, and a method for finding a hyperplane so as to allow erroneous determination of the presence or absence of liver cancer is called a soft-margin support vector machine.

The neural network is a mathematical model of nerve cells (neurons) in the human brain and their connections. The neural network includes an input layer, an output layer, and a hidden layer. A weight indicating the strength of a connection between neurons is provided between the respective layers. In learning of the neural network, using the data with known determination (the presence or absence of liver cancer), the weight is adjusted such that the presence or absence of liver cancer can be correctly determined in the output layer.

The random forest is an ensemble learning algorithm obtained by integrating a plurality of decision trees. The decision trees are tree-like models created by finding the explanatory variable that affects the objective variable.

As shown in FIG. 2, the trained model generator 11 generates the trained model information M that allows separation of the biological information about the patients P1 (patient group PF) who have liver cancer from the biological information about the patients P1 (patient group PF) who do not have liver cancer. For example, in FIG. 2, biological information (I1) located above the trained model information M is the biological information about the patients P1 who have liver cancer, and biological information (I2) located below the trained model information M is the biological information about the patients P1 who do not have liver cancer. Furthermore, machine learning software (application) is written in a programming language such as R language, for example. The trained model information M includes R language objects. These objects do not include the personal information about the patients P1.

The diagnosis assistance device 100a is configured to export the trained model information M generated by the trained model generator 11 from the trained model generator 11 via an external network 30. That is, the diagnosis assistance device 100a is configured to output the trained model information M in a format that can be read by the determiner 21.

In the first embodiment, the determiner 21 receives the trained model information M generated by the trained model generator 11 via the external network 30. Furthermore, the determiner 21 is configured to determine the presence or absence of a disease in a patient P2 who is not included in the patient group PF based on the received trained model information M. The trained model information M is exported from the trained model generator 11 and imported into the determiner 21 via the external network 30.

Specifically, the electronic clinical record database 20 of the facility 2 stores the electronic clinical record data of the patient P2. The patient P2 is a patient P2 who is not included in the patients P1 (patient group PF) having the biological information used when the trained model information M s generated. The electronic clinical record data of patient P2 includes the biological information (data on HCV antibody, HBs antigen, age, gender, height, weight, albumin, total bilirubin, AST, ALT, ALP, GGT, platelets, AFP, L3 fractions, and, DCP) stored in the above electronic clinical record database 10 of the facility 1. The electronic clinical record data of the patient P2 does not include the presence or absence of liver cancer.

The determiner 21 is configured to determine the presence or absence of liver cancer in the patient P2, who is not included in the patient group PF, based on the trained model information M received via the external network 30. Specifically, the biological information included in the electronic clinical record data of the patient P2 is input to the trained model information M. Then, in FIG. 2, the presence or absence of liver cancer is determined depending on whether the biological information (I3) about the patient P2 is classified above or below the trained model information M.

### Experiment

A machine learning experiment based on patient's biological information is now described.

In this experiment, trained model information was generated based on biological information about 1,584 patients who visited the hospital as outpatients. The patient's biological information includes data on the presence or absence of liver cancer, HCV antibody, HBs antigen, age, gender, height, weight, albumin, total bilirubin, AST, ALT, ALP, GGT, platelets, AFP, L3 fractions, and DCP. In addition, a logistic regression, a soft-margin support vector machine, a neural network, a random forest, etc. were used as machine learning algorithms. Furthermore, cross-validation is performed to determine the correctness of machine learning. In cross-validation, the biological information about 1,582 patients is divided, trained model information is generated from a portion of the biological information, and the correct answer rate is obtained from the remaining portion. Consequently, it has been confirmed that the correct answer rate of around 80% or more than 80% can be obtained in any of the machine learning algorithms. Thus, for example, a machine learning algorithm having the correct answer rate of more than 80% is selected such that it becomes possible to determine the presence or absence of liver cancer using the trained model information M with relatively high accuracy.

### Advantages of First Embodiment

In the first embodiment, the following advantages are obtained.

In the first embodiment, as described above, the diagnosis assistance system 100 includes the determiner 21 configured to receive the trained model information M generated by the trained model generator 11 via the external network 30 and to determine the presence or absence of a disease (liver cancer) in the patient P2 who is not included in the patient group PF based on the received trained model information M. Accordingly, the learn model information M can be received by the determiner 21 installed in a small hospital or the like via the external network 30, and thus even in a small hospital (facility 2) or the like in which it is difficult to acquire data for machine learning, the presence or absence of a disease in the patient P2 can be determined based on the trained model information M. Furthermore, the trained model information M generated by the trained model generator 11 includes statistical information that does not include the personal information about the patients P1, for example. Thus, even when the trained model information M is provided to the outside (such as a small hospital) via the external network 30, the personal information about the patients P1 is not leaked. Consequently, even in a small hospital in which it is difficult to acquire data for machine learning, for example, the personal information about the patients P1 is not leaked to the outside (such as a small hospital), and the presence or absence of a disease in the patient P2 can be determined based on the result (trained model information M) of machine learning.

In the first embodiment, as described above, the trained model generator 11 is configured to extract the biological information about the individual patients P1 from the electronic clinical record data stored in the electronic clinical record database 10 and to generate the trained model information M based on the extracted biological information about the individual patients P1. Accordingly, the trained model information M is generated based only on the biological information about the individual patients P1 extracted from the electronic clinical record data without using the identification information about the patients P1, and thus leakage of the personal information about the patients P1 can be reliably prevented.

In the first embodiment, as described above, the trained model information M is exported from the trained model generator 11 and imported into the determiner 21 via the external network 30. Accordingly, even when the application of the trained model generator 11 and the application of the determiner 21 are different from each other, the trained model information M is output (exported) from the trained model generator 11 in a format that can be read by the application of the determiner 21 such that the trained model information M can be used in the determiner 21.

In the first embodiment, as described above, the biological information about the patient group PF includes the data on the presence or absence of liver cancer, HCV antibody, HBs antigen, age, gender, height, weight, albumin, total bilirubin, AST, ALT, ALP, GGT, platelets, AFP, L3 fractions, and DCP, and the determiner 21 is configured to determine the presence or absence of liver cancer in the patient P2, who is not included in the patient group PF, based on the trained model information M received via the external network 30. Accordingly, as described in the above experiment, the presence or absence of liver cancer in the patient P2 can be determined with a relatively high correct answer rate (a correct answer rate of around 80% or more than 80%).

### Second Embodiment

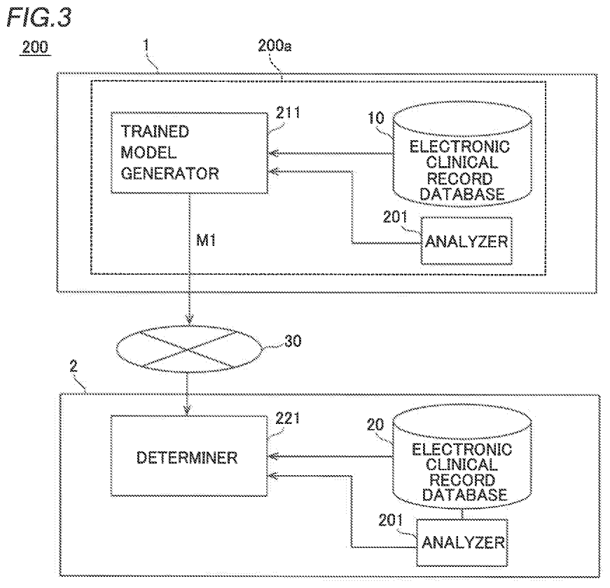

The configuration of a diagnosis assistance system 200 according to a second embodiment is now described with reference to FIG. 3. In the second embodiment, trained model information M1 is generated based on analytical information about biological samples of patients P1.

In the diagnosis assistance system 200 (diagnosis assistance device 200a), a facility 1 includes an analyzer 201 configured to analyze the biological samples of patients P1. The analyzer 201 is a mass spectrometer, for example. Furthermore, the analyzer 201 is configured to identify a molecule that serves as a marker for a disease (liver cancer) in the patients P1, for example. The analytical information about the analyzed biological samples of the patients P1 is automatically associated with (automatically linked to) electronic clinical record data.

In the second embodiment, a trained model generator 211 is configured to generate the trained model information M1 based on biological information about the individual patients P1 extracted from the electronic clinical record data stored in an electronic clinical record database 10 and the analytical information about the biological samples of the individual patients P1 associated with the electronic clinical record data. That is, the trained model information M1 reflects the biological information about the patients P1 and the analytical information about the biological samples, and in machine learning of the trained model generator 211, the information amount (feature amount) of training data is larger as compared with the first embodiment.

A facility 2 includes the analyzer 201 configured to analyze a biological sample of a patient P2. Analytical information about the biological sample of the patient P2 analyzed by the analyzer 201 is associated with electronic clinical record data of the patient P2. A determiner 221 determines the presence or absence of a disease in the patient P2, who is not included in a patient group PF, based on the trained model information M1 received via an external network 30. Specifically, biological information included in the electronic clinical record data of the patient P2 and the analytical information associated with the electronic clinical record data are applied (input) to the trained model information M1. Thus, the presence or absence of liver cancer is determined.

### Advantages of Second Embodiment

In the second embodiment, the following advantages are obtained.

In the second embodiment, as described above, the trained model generator 211 is configured to generate the trained model information M1 based on the biological information about the individual patients P1 extracted from the electronic clinical record data stored in the electronic clinical record database 10 and the analytical information about the biological samples of the individual patients P1 associated with the electronic clinical record data. Accordingly, the trained model information M1 is generated based on the analytical information about the biological samples of the individual patients P1 in addition to the biological information about the individual patients P1, and thus the presence or absence of a disease in the patient P2 can be more accurately determined.

### Third Embodiment

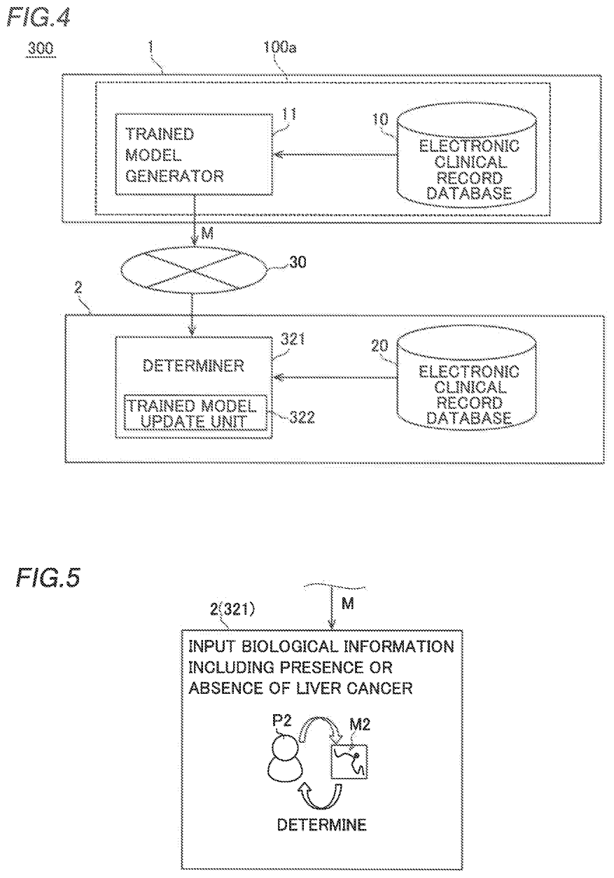

The configuration of a diagnosis assistance system 300 according to a third embodiment is now described with reference to FIGS. 4 and 5. In the third embodiment, trained model information M is updated based on biological information about a patient P2.

In the third embodiment, a determiner 321 of the diagnosis assistance system 300 includes a trained model update unit 322. The trained model update unit 322 is configured to update the trained model information M received via the external network 30 based on the biological information about the patient P2 with a known presence or absence of a disease, who is not included in a patient group PF. Machine learning is performed on all the biological information about the patient group PF such that the trained model information M is generated. The trained model update unit 322 updates the trained model information M based only on the biological information about the patient P2 without using the biological information about the patient group PF to generate trained model information M2. Thus, the biological information about the patient P2 is reflected in the trained model information M2. For example, parameters of the trained model information M2 are updated based on the biological information about the patient P2. The trained model update unit 322 is an example of a “first trained model update unit” in the claims.

### Advantages of Third Embodiment

In the third embodiment, the following advantages are obtained.

In the third embodiment, as described above, the determiner 321 includes the trained model update unit 322 configured to update the trained model information M received via the external network 30 based on the biological information about the patient P2 with a known presence or absence of a disease, who is not included in the patient group PF. Accordingly, even when it is difficult to acquire data for machine learning, the quality (determination ability) of the trained model information M2 can be improved based on the biological information about the patient P2 with a known presence or absence of a disease, who is not included in the patient group PF.

### Fourth Embodiment

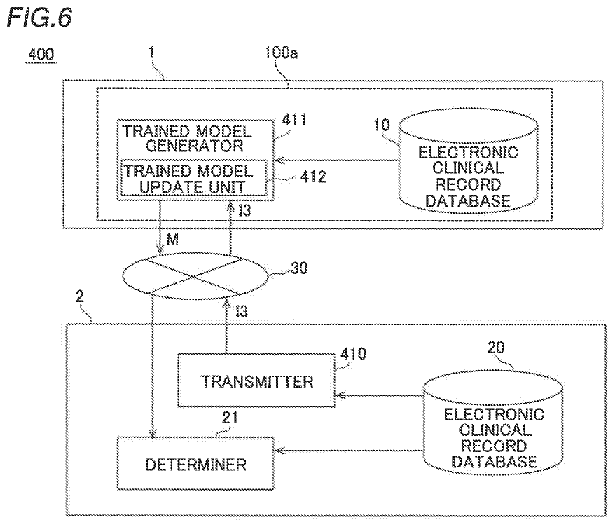

The configuration of a diagnosis assistance system 400 according to a fourth embodiment is now described with reference to FIG. 6. In the fourth embodiment, a transmitter 410 is provided to transmit biological information I3 about a patient P2.

In the fourth embodiment, the diagnosis assistance system 400 includes the transmitter 410. The transmitter 410 is provided in a facility 2. The transmitter 410 is configured to transmit the biological information I3 about the patient P2 with a known presence or absence of a disease, who is not included in a patient group PF, to a trained model generator 411 via an external network 30 in a state in which identification information about the patient P2 has been removed from the biological information I3. Specifically, the biological information I3 about each patient P2 is extracted from electronic clinical record data stored in an electronic clinical record database 20 of the facility 2. The identification information (personal information such as a name) about the patient P2 is not extracted. The transmitter 410 transmits the extracted biological information I3 about each patient P2 to the trained model generator 411.

The trained model generator 411 includes a trained model update unit 412. The trained model update unit 412 is configured to update trained model information M based on the biological information I3 about the patient P2 with a known presence or absence of a disease, who is not included in the patient group PF. The trained model update unit 412 updates the trained model information M based only on the biological information I3 about the patient P2 without using biological information about the patient group PF. The biological information I3 includes data on the presence or absence of liver cancer, HCV antibody, HBs antigen, age, gender, height, weight, albumin, total bilirubin, AST, ALT, ALP, GGT, platelets, AFP, L3 fractions, and DCP. The transmitter 410 may periodically transmit the biological information I3 about the patient P2 to the trained model generator 11. Thus, the trained model information M is periodically updated. The trained model update unit 412 is an example of a “second trained model update unit” in the claims.

### Advantages of Fourth Embodiment

In the fourth embodiment, the following advantages are obtained.

In the fourth embodiment, as described above, the transmitter 410 is provided to transmit the biological information I3 about the patient P2 with a known presence or absence of a disease, who is not included in the patient group PF, to the trained model generator 11 via the external network 30 in a state in which the identification information about the patient P2 has been removed from the biological information I3. Accordingly, the quality (determination ability) of the trained model information M can be improved by the trained model update unit 412 based on the biological information I3 about the patient P2 with a known presence or absence of a disease, who is not included in the patient group PF, transmitted from the outside (facility 2) via the external network 30. Consequently, the trained model information M with improved quality (determination ability) is received by a plurality of facilities 2 again via the external network 30 such that the presence or absence of a disease in a patient (a patient with an unknown presence or absence of a disease) can be determined based on the common trained model information M with improved quality in the plurality of facilities 2. Furthermore, the transmitter 410 transmits the biological information I3 about the patient P2 to the trained model update unit 412 in a state in which the identification information about the patient P2 has been removed from the biological information I3, and thus the identification information about the patient P2 is not leaked to the outside (such as a facility 1).

### MODIFIED EXAMPLES

The embodiments disclosed this time must be considered as illustrative in all points and not restrictive. The scope of the present invention is not shown by the above description of the embodiments but by the scope of claims for patent, and all modifications (modified examples) within the meaning and scope equivalent to the scope of claims for patent are further included.

For example, while the example in which machine learning is performed on the biological information about the individual patients extracted from the electronic clinical record data has been shown in each of the aforementioned first to fourth embodiments, the present invention is not limited to this. For example, machine learning may be performed based on the biological information about the individual patients other than the electronic clinical record data.

While the example in which the presence or absence of liver cancer is determined based on the trained model information has been shown in each of the aforementioned first to fourth embodiments, the present invention is not limited to this. The present invention can also be applied to determination of a disease (such as pancreatic cancer) other than liver cancer.

While the example in which as the biological information about the patient group, all of the data on HCV antibody, HBs antigen, age, gender, height, weight, albumin, total bilirubin, AST, ALT, ALP, GGT, platelets, AFP, L3 fractions, and DCP is used has been shown in each of the aforementioned first to fourth embodiments, the present invention is not limited to this. For example, as the biological information about the patient group, some of the data on HCV antibody, HBs antigen, age, gender, height, weight, albumin, total bilirubin, AST, ALT, ALP, GGT, platelets, AFP, L3 fractions, and DCP may be used.

While in this specification, the second embodiment in which the trained model information is generated based on the biological information extracted from the electronic clinical record data and the analytical information about the biological samples and the third and fourth embodiments in which the trained model information is updated are described as separate embodiments, the configuration of the second embodiment, the configuration of the third embodiment, and the configuration of the fourth embodiment may be combined.

While the example in which the logistic regression, the soft-margin support vector machine, the neural network, and the random forest are used as machine learning algorithms has been shown in each of the aforementioned first to fourth embodiments, the present invention is not limited to this. For example, machine learning algorithms other than the logistic regression, the soft-margin support vector machine, the neural network, and the random forest may be used.

### DESCRIPTION OF REFERENCE NUMERALS

10: electronic clinical record database (storage)

11, 211, 411: trained model generator

21, 221, 321: determiner

30: external network

100, 200, 300, 400: diagnosis assistance system

100a, 200a: diagnosis assistance device

322: trained model update unit (first trained model update unit)

401: transmitter

412: trained model update unit (second trained model update unit)

M, M1, M2: trained model information

P1, P2: patient

PF: patient group

1. A diagnosis assistance system comprising:
a storage configured to store biological information about a patient group;
a trained model generator configured to generate trained model information derived from a pattern included in the biological information about the patient group, by machine learning based on the biological information about the patient group stored in the storage; and
a determiner configured to receive the trained model information generated by the trained model generator via an external network, the determiner being configured to determine, based on the received trained model information, a presence or absence of a disease in a patient with an unknown disease state.

2. The diagnosis assistance system according to claim 1, wherein
the storage is configured to store electronic clinical record data with identification information about an individual patient included in the patient group and the biological information about the individual patient being described therein; and
the trained model generator is configured to extract the biological information about the individual patient from the electronic clinical record data stored in the storage, and to generate the trained model information based on the extracted biological information about the individual patient.

3. The diagnosis assistance system according to claim 2, wherein the trained model generator is configured to generate the trained model information based on the biological information about the individual patient extracted from the electronic clinical record data stored in the storage, and analytical information about a biological sample of the individual patient associated with the electronic clinical record data.

4. The diagnosis assistance system according to claim 1, wherein the determiner includes a first trained model update unit configured to update the trained model information received via the external network based on the biological information about a patient with a known presence or absence of the disease, who is not included in the patient group.

5. The diagnosis assistance system according to claim 1, further comprising:
a transmitter configured to transmit the biological information about a patient with a known presence or absence of the disease, who is not included in the patient group, to the trained model generator via the external network in a state in which identification information about the patient has been removed from the biological information, wherein
the trained model generator includes a second trained model update unit configured to update the trained model information based on the biological information transmitted from the transmitter.

6. The diagnosis assistance system according to claim 1, wherein the trained model information is exported from the trained model generator and imported into the determiner via the external network.

7. The diagnosis assistance system according to claim 1, wherein
the biological information about the patient group includes data on a presence or absence of liver cancer, HCV antibody, HBs antigen, age, gender, height, weight, albumin, total bilirubin, AST, ALT, ALP, GGT, a platelet, AFP, an L3 fraction, and DCP; and
the determiner is configured to determine the presence or absence of liver cancer in the patient with the unknown disease state based on the trained model information received via the external network.

8. A diagnosis assistance device comprising:
a storage configured to store biological information about a patient group; and
a trained model generator configured to generate trained model information derived from a pattern included in the biological information about the patient group, by machine learning based on the biological information about the patient group stored in the storage; wherein
the trained model information generated by the trained model generator is exported to an outside via an external network.

9. The diagnosis assistance device according to claim 8, wherein
the storage is configured to store electronic clinical record data with identification information about an individual patient included in the patient group and the biological information about the individual patient being described therein; and
the trained model generator is configured to extract the biological information about the individual patient from the electronic clinical record data stored in the storage, and to generate the trained model information based on the extracted biological information about the individual patient.

10. The diagnosis assistance device according to claim 9, wherein the trained model generator is configured to generate the trained model information based on the biological information about the individual patient extracted from the electronic clinical record data stored in the storage, and analytical information about a biological sample of the individual patient associated with the electronic clinical record data.

11. A diagnosis assistance method comprising:
generating trained model information derived from a pattern included in biological information about a patient group, by machine learning based on the biological information about the patient group stored in a storage; and
receiving the generated trained model information via an external network and determining, based on the received trained model information, a presence or absence of a disease in a patient with an unknown disease state.

12. A diagnosis assistance method comprising:
generating trained model information derived from a pattern included in biological information about a patient group, by machine learning based on the biological information about the patient group stored in a storage; and
exporting the generated trained model information to an outside via an external network.

13. A diagnosis assistance method comprising:
generating trained model information derived from a pattern included in biological information about a patient group, by machine learning based on the biological information about the patient group stored in a storage;
exporting the generated trained model information to an outside via an external network; and
receiving the generated trained model information via the external network and determining, based on the received trained model information, a presence or absence of a disease in a patient with an unknown disease state.
